(19) United States
(12) Patent Application Publication
(10) Pub. No.: US 2014/0155763 A1
(43) Pub. Date: Jun. 5, 2014

(54) MEDICAL ANALYSIS AND DIAGNOSTIC SYSTEM

(71) Applicant: Ben F. Bruce, Arlington, TX (US)

(72) Inventor: Ben F. Bruce, Arlington, TX (US)

(21) Appl. No.: 14/094,579

(22) Filed: Dec. 2, 2013

Related U.S. Application Data
(60) Provisional application No. 61/797,206, filed on Dec. 3, 2012.

(51) Int. Cl. A61B 5/00; A61B 5/0205

(52) U.S. Cl. CPC A61B 5/7221; A61B 5/02055; A61B 5/74
USPC 600/484

(57) ABSTRACT

A computerized method includes diagnosing a patient. The diagnosing includes receiving a patient identification of the patient. The diagnosing includes determining, using one or more sensors, one or more current body characteristics of the patient. The diagnosing includes creating a current multimedia representation for each of the one or more current body characteristics determined by using the sensor. The diagnosing includes comparing the current multimedia representation to previous multimedia representations of each of the one or more body characteristics from other persons. The diagnosing includes selecting a diagnosis and a diagnosis confidence factor for the diagnosis for the patient based on the comparing of the current multimedia representation to the previous multimedia representations of each of one or more the body characteristics. The diagnosing includes determining whether the diagnosis confidence factor exceeds a high confidence factor threshold.

### RELATED APPLICATIONS(S)

This patent application claims the benefit of priority to U.S. Provisional Patent Application Ser. No. 61/797,206, filed on Dec. 3, 2012, which is incorporated herein by reference.

### COPYRIGHT

A portion of the disclosure of this document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software, data, and/or screenshots which may be described below and in the drawings that form a part of this document: Copyright©2013, Trinity Technical Group, Inc. All Rights Reserved.

### TECHNICAL FIELD

The present invention relates generally to the field of medical examination, evaluation, triage, diagnosis and treatment, and more particularly to a method, system and program for making specific and unambiguous, or high confidence informed decisions on the diagnosis of medical and trauma conditions using analog, digital and/or digitizing sensors, and inputs from various interfaces to gather patient information that is then processed, analyzed, classified, characterized, recognized and compared with historical patient data if available in order to generate search criteria suitable for use with a diagnostic search engine. Expert systems, state machines or other methodologies may implemented as a diagnostic search engine or engines and such diagnostic search engines should utilize all available search criteria derived from the collected and processed patient data, vital signs, symptoms and historical data, if available, to search a diagnostic database and make a unique and unambiguous diagnosis or a high confidence informed decision on a diagnosis of an illness, malady, disease, infection, condition or trauma afflicting the patient. In the event that a unique and unambiguous diagnosis or a high confidence informed decision on a diagnosis cannot be made based upon the collected patient data, signs and symptoms, the system may recommend additional testing that will aid in producing a unique and unambiguous diagnosis or a high confidence informed decision on a diagnosis with as few tests as possible. In the event that the diagnosis remains ambiguous, the system may refer the patient to a medical doctor or specialist for further treatment. Once a diagnosis is finalized, the system should have the capability to look up the recommended treatment regime associated with the diagnosis along with any associated prescription or non-prescription pharmaceuticals. Finally, the system may print off hard copies of the diagnosis and treatment regime, and print out a list of any associated non-prescription pharmaceuticals and/or prescriptions for any prescription pharmaceuticals. The system will then save all current patient data into the patient's file for future reference.

### BACKGROUND

The approaches described in this section could be pursued, but are not necessarily approaches that have been previously conceived or pursued. Therefore, unless otherwise indicated herein, the approaches described in this section are not prior art to the claims in this application and are not admitted to be prior art by inclusion in this section.

The collection of medical patient signs, symptoms and data, analysis of these signs, symptoms and data, diagnosis of medical conditions, and determination of curative treatment has traditionally been provided by medical doctors or specialists who have been through many years of specialized education, training and experience.

A number of devices are available to these medical doctors for use in collecting patient data which can be used to help make them make an informed decision on a diagnosis of the specific illness, malady, disease, infection, condition or trauma afflicting the patient. Among other things, these devices may include scales, thermometers, stethoscopes, sphygmomanometers, and otoscopes. Once the patient's chief complaint has been identified and other patient information gathered, these devices can be used to collect pertinent patient signs, symptoms and data that the medical doctor or specialist may utilize, along with his or her own personal education, training, experience, memory and cognitive skills to make an informed decision on a diagnosis and recommend curative treatment regimes which may or may not include prescription or over-the-counter pharmaceuticals.

Additional laboratory testing including, but not limited to, blood tests, urinalysis, cultures, electrocardiogram (ECG or EKG), Sonogram/Ultrasounds, X-rays, Computerized Axial Tomography (CAT) Scans, Magnetic Resonance Imaging (MRI) or Positron Emission Tomography (PET) Scans may also be required in order to more definitively identify the illness, malady, disease, infection and/or trauma conditions affecting the patient.

Notes related to patient data, examination, diagnosis, treatment and pharmaceuticals prescribed are normally written by hand and copies, if any, are put into a patient file which is physically stored in the local facility. Some associated test results such as blood tests, urinalysis and electrocardiogram (ECG or EKG) may be printed out in hard copy and may be cross referenced to or included in the patient's file as well. Results of other tests such as Sonogram/Ultrasounds, X-rays, Computerized Axial Tomography (CAT) Scans, Magnetic Resonance Imaging (MRI) or Positron Emission Tomography (PET) Scans may be recorded in other media types and may be stored locally or in other facilities and may or may not be cross-referenced to the patient for future reference.

### SUMMARY

In some example embodiments, a computerized method includes diagnosing a patient. The diagnosing includes receiving a patient identification of the patient. The diagnosing includes determining, using one or more sensors, one or more current body characteristics of the patient comprising at least one of pulse rate, body temperature, blood pressure, respiration, and skin condition. The diagnosing includes creating a current multimedia representation for each of the one or more current body characteristics determined by using the sensor. The diagnosing includes comparing the current multimedia representation to previous multimedia representations of each of the one or more body characteristics from other persons. The diagnosing includes selecting a diagnosis and a diagnosis confidence factor for the diagnosis for the patient based on the comparing of the current multimedia representation to the previous multimedia representations of each of one or more the body characteristics. The diagnosing includes determining whether the diagnosis confidence factor exceeds a high confidence factor threshold. The diagnosing includes in response to the diagnosis confidence factor not exceeding the high confidence factor threshold, selecting a different current body characteristic of the patient to determine to increase the diagnosis confidence factor. The diagnosing includes in response to the diagnosis confidence factor exceeding the high confidence factor threshold, selecting the diagnosis for the patient.

### BRIEF DESCRIPTION OF THE DRAWINGS

The embodiments are provided by way of example and not limitation in the figures of the accompanying drawings, in which like references indicate similar elements and in which:

FIG. 1 is a system diagram for a medical analysis and diagnostic system, according to some example embodiments.

FIG. 2 is a system diagram for possible use in a standalone mobile or facility environment, according to some example embodiments.

FIG. 3 is a system diagram for possible use in a facility or remote distributed (client/server) environment, according to some example embodiments.

FIG. 4 is a system diagram for possible use in a facility or remote distributed (client/server) environment, according to some example embodiments.

FIG. 5 is a diagram of a method for main processing in a Medical Analysis and Diagnostic System, according to some example embodiments.

FIG. 6 is a diagram of a method for a diagnostic mode in a Medical Analysis and Diagnostic System, according to some example embodiments.

FIG. 7 is a diagram of a method for a monitor mode in a Medical Analysis and Diagnostic System, according to some example embodiments.

FIG. 8 is a diagram of a method for a physical examination mode in a Medical Analysis and Diagnostic System, according to some example embodiments.

FIG. 9 is a diagram of a method for a treatment determination mode in a Medical Analysis and Diagnostic System, according to some example embodiments.

FIG. 10 is a diagram of a method for a continuation of the diagnostic mode in a Medical Analysis and Diagnostic System, according to some example embodiments.

FIG. 11 is a diagram of a method for a maintenance mode in a Medical Analysis and Diagnostic System, according to some example embodiments.

FIG. 12 is a diagram of a method for sensor operation verification in a Medical Analysis and Diagnostic System mode, according to some example embodiments.

### DETAILED DESCRIPTION

Methods, apparatus and systems for a medical analysis and diagnostic system are described. In the following description, numerous specific details are set forth. However, it is understood that embodiments of the invention may be practiced without these specific details. In other instances, structures and techniques have not been shown in detail in order not to obscure the understanding of this description.

Some example embodiments may utilize a mobile computer system with specialized hardware, firmware, software and databases, and a basic sensor suite such as, but not limited to analog, digital or digitizing sensors such as scales, stethoscopes, thermometers, sphygmomanometers, perfusion oxygen or hematocrit saturation monitors, ophthalmoscopes, funduscopes, and otoscopes to gather patient information such as weight, pulse rate, pulse characterization and pattern recognition, respiration rate, respiration and body sounds characterization and pattern recognition, body temperature, blood pressure, oxygen saturation, perfusion, skin temperature, skin moisture level, electrocardiogram, imaging and/or video of eyes, ears, nose and throat, and imaging and/or video for skin, scalp and extremities to collect data to be transmitted to and processed by the mobile system. In some embodiments, such sensors can collect analog, digital, discrete, pressure, audio, high definition color and/or grayscale image and video, and/or other data types and convert this data to a format suitable for uploading to the mobile computer system for further processing, analyzing, classifying, characterizing, image and/or pattern recognition, comparing and generating search criteria suitable for use with the diagnostic search engine. One or more expert systems, state machines or other methodologies may implemented as a diagnostic search engine or engines and such diagnostic search engines should utilize all available search criteria derived from the collected patient data, signs, symptoms and historical data, if available, to search the diagnostic database and make a unique and unambiguous diagnosis or a high confidence informed decision on a diagnosis of an illness, malady, disease, infection, condition or trauma afflicting the patient. In the event that a unique and unambiguous diagnosis or a high confidence informed decision on a diagnosis cannot be made based upon the collected patient data, signs and symptoms, the system may recommend additional testing that will aid in producing a unique and unambiguous diagnosis or a high confidence informed decision on a diagnosis with as few tests as possible. In the event that the diagnosis remains ambiguous, the system may refer the patient to a medical doctor or specialist for further treatment. Once a diagnosis is finalized, the system should have the capability to look up the recommended treatment regime associated with the diagnosis along with any associated prescription or non-prescription pharmaceuticals. Finally, the system may print off hard copies of the diagnosis and treatment regime, and print out a list of any associated non-prescription pharmaceuticals and/or prescriptions for any prescription pharmaceuticals. The system will then save all current patient data into the patient's file for future reference. Such mobile systems could be easily transported to or utilized in urban or remote areas which have emergency medical requirements or that are underserved by trained medical doctors and specialists. Such systems could provide medical and trauma related diagnostic services equivalent to a general practitioner or family doctor in an office environment.

When connected to LAN, WAN, wireless, cellular or other network services, such mobile systems should be able to download and utilize any existing and available prior patient analog, digital, discrete, pressure, image, video, audio or other media inputs or files along with patient digital discrete, pressure, image, video, audio or other media inputs or files from results from more sophisticated laboratory and test equipment such as, but not limited to, blood tests, urinalysis, cultures, x-ray machines, contact or non-contact tonometry, Sonogram/Ultrasound, Electrocardiogram, Computerized Axial Tomography (CAT) scans, Magnetic Resonance Imaging (MRI) and Positron Emission Tomography (PET) scans which may be processed, analyzed, classified, recognized and/or characterized to identify any signs, symptoms, potential anomalies or abnormal characteristics and produce search criteria suitable for use in the diagnostic search engine.

Other example embodiments might include dedicated or client/server systems in fixed locations that are capable of servicing multiple clients in one or more local or remote locations. Such systems may utilize specialized hardware, firmware, software and databases in the server systems while the client systems might utilize a basic sensor suite such as, but not limited to analog, digital or digitizing sensors such as scales, stethoscopes, thermometers, sphygmomanometers, perfusion oxygen or hematocrit saturation monitors, ophthalmoscopes, funduscopes, and otoscopes to gather patient information such as weight, pulse rate, pulse characterization, respiration rate, respiration and body sounds characterization, body temperature, blood pressure, oxygen saturation, perfusion, skin temperature, skin moisture level, electrocardiogram, high definition color and/or grayscale imaging and/or video of eyes, ears, nose, throat, skin, scalp and extremities to collect data to be transmitted to and processed by the server system. Such sensors should be capable of collecting analog, digital, discrete, pressure, audio, high definition color and/or grayscale image and video, and/or other data types and converting this data to a format suitable for uploading to the mobile computer system for further processing, analyzing, classifying, characterizing, recognizing, comparing and generating search criteria suitable for use with the diagnostic search engine. One or more expert systems, state machines or other methodologies may implemented as a diagnostic search engine or engines and such diagnostic search engines should utilize all available search criteria derived from the collected and processed patient data, signs, symptoms and historical data, if available, to search the diagnostic database and make a unique and unambiguous diagnosis or a high confidence informed decision on a diagnosis of an illness, malady, disease, infection, condition or trauma afflicting the patient. In the event that a unique and unambiguous diagnosis or a high confidence informed decision on a diagnosis cannot be made based upon the collected patient signs, data and symptoms, the system will recommend additional tests that should produce a unique and unambiguous diagnosis or an informed decision on a diagnosis with the fewest number of tests possible. In the event that the diagnosis remains ambiguous, the system will refer the patient to a medical doctor or specialist for further treatment. Once a diagnosis is finalized, the system should have the capability to look up the recommended treatment regime associated with the diagnosis along with any associated prescription or non-prescription pharmaceuticals. Finally, the system may have the capability to print off hard copies of the diagnosis and treatment regime, and print out a list of any associated non-prescription pharmaceuticals and/or prescriptions for any prescription pharmaceuticals. The system will then save all patient data into the patient's file for future reference. Such client/server systems could provide medical and trauma related diagnostic services equivalent to a general practitioner or family doctor in a hospital environment.

Other example embodiments might include the capability to directly interface with and/or input patient analog, digital, discrete, pressure, image, video, audio or other media inputs or files from more sophisticated laboratory and test equipment such as blood tests, urinalysis, cultures, x-ray machines, contact or non-contact tonometry, Sonogram/Ultrasound, Electrocardiogram, Computerized Axial Tomography (CAT) scans, Magnetic Resonance Imaging (MRI) and Positron Emission Tomography (PET) scans, which may be processed, analyzed, classified, recognized and/or characterized to identify any signs, symptoms, potential anomalies or abnormal characteristics and produce search criteria suitable for use in the diagnostic search engine.

Other example embodiments might include a touch screen, keyboard or other manual inputs for operator identification and verification, patient name and personal information, insurance, medical information including, but not limited to age, height, weight, known conditions, known drug allergies, current prescriptions, etc., and other information as required. Touch screen, keyboard or other manual inputs may also be used to input the Chief Complaint(s) and input answers to predetermined lists of questions based upon whether the patient has a trauma or is suffering from a medical condition. Finally, touch screen, keyboard or other manual inputs may be utilized to enter manual results or operator observed results including but not limited to rebound tenderness, swelling, joint swelling, joint displacement, etc.

Other example embodiments of the present invention might include specialized audio processing, image processing, video processing and other processing types that may be used along with image and pattern recognition algorithms, all of which may be implemented in hardware, firmware, software or any combination thereof.

Other example embodiments might include processing, analyzing, classifying, recognizing, characterizing and/or comparing any available analog, digital, discrete, pressure, image, video, audio or other media inputs by hardware, firmware or software to identify any signs, symptoms, potential anomalies or abnormal characteristics and produce search criteria suitable for use in the diagnostic search engine.

Other example embodiments might include processing, analyzing, classifying, recognizing, characterizing and comparing any available currently available and/or historical inputs or other media files such as, but not limited to age, sex, body weight, pulse rate, respiration rate, body temperature, blood pressure, oxygen saturation, skin temperature and moisture level, and perfusion being processed, analyzed, classified, correlated, recognized, characterized and/or compared in order to identify any vital signs, symptoms, potential anomalies or abnormal characteristics and produce search criteria suitable for use in the diagnostic search engine.

Other example embodiments might include currently available and/or historical audio, pressure or other inputs or media files being processed, analyzed, classified, correlated, recognized, characterized and/or compared with respect to heartbeat characterization and pattern recognition, pulse characterization and pattern recognition, respiration, breathing and other body sounds in order to identify any signs, symptoms, potential anomalies or abnormal characteristics and produce search criteria suitable for use in the diagnostic search engine.

Other example embodiments might include currently available and historical image or video inputs or other media files being processed, analyzed, classified, recognized, characterized and/or compared with respect to signs or symptoms including but not limited to pupil size and relative pupil size; pupil reaction to light; eye conditions including, but not limited to conjunctivitis (pink eye), uveitis, iritis, scleritis, keratitis and stye (bump on the eye); ear canal and ear drum; nasal passages; throat; skin medical conditions including, but not limited to rashes, blisters, ulcers, acne, eczema, ringworm, psoriasis, scabies, shingles, psoriasis, rosacea, basal cell carcinoma, squamous cell carcinoma, and melanoma; skin trauma conditions including, but not limited to contusions (bruises), abrasions (scrapes), lacerations (cuts, scratches or punctures), burns (chemical or heat); serious skin trauma conditions; nail conditions including, but not limited to hangnail, fungus, ingrown nail; scalp or hair conditions including, but not limited to alopecia, head lice, dandruff, ingrown hair; and any other items of interest such as, but not limited to swellings, joint swelling or joint displacement; internal medical conditions including but not limited to tumors, growths, cysts, cancers, aneurysms, hernias, broken or dislocated bones and any other medical issues in order to identify any signs, symptoms, potential anomalies or abnormal characteristics and produce search criteria suitable for use in the diagnostic search engine.

Other example embodiments might include currently available discrete, pressure, image, video, audio or other inputs or media files being processed, analyzed, classified, recognized, characterized, compared and correlated with historical discrete, image, video, audio or other media files to do a comparative analysis in order to identify any differences, signs, symptoms, potential anomalies, abnormal characteristics and/or trends, and produce search criteria suitable for use in the diagnostic search engine.

Other example embodiments might include the implementation of a diagnostic search engine or engines as expert systems, state machines or other methodologies that utilize currently available geographic and point in time information, patient chief complaint(s), patient interviews, search criteria generated from patient basic sensor data, search criteria generated from patient advanced sensor data and search criteria generated from patient historical data to produce a unique and unambiguous diagnosis or a high confidence informed decision on a diagnosis, the best treatment regimen associated with that diagnosis and which over-the-counter or prescription pharmaceuticals, if any, should be prescribed as part of the treatment regimen for the patient's medical or trauma condition, without the participation or intervention of a medical doctor.

Another example embodiment of the present invention provides a methodology wherein if a unique and unambiguous diagnosis or a high confidence informed decision on a diagnosis cannot be obtained with the available patient information and data, the diagnostic engine should produce a list of possible diagnoses with confidence factors for each one and based upon the current circumstances and available patient data, the system will either select the highest probability diagnosis consistent with approved medical protocols, recommend additional testing or refer the patient to a medical doctor or specialist for further treatment. In the event that additional testing is required to finalize a diagnosis, specific tests should be recommended in an order designed to minimize the amount of testing required and data acquisition interfaces are provided to accept these test results as they become available.

Another illustrated embodiment of the present invention provides a methodology to standardize patient interviews, data collection, diagnostics, treatment regimens and dispensing of prescriptions according to defined and previously approved guidelines.

Another illustrated embodiment of the present invention provides a methodology for sharing patient medical information via cellular, wireless, Local Area Network (LAN) and Wide Area Network (WAN) connectivity and using that information from different sources to improve the patient's diagnostic results and resulting health care.

Another illustrated embodiment of the present invention provides a methodology for processing, analyzing, classifying, correlating, recognizing, characterizing and/or comparing multiple patient signs, symptoms, and/or diagnoses based on geographic areas to determine if there is a potential for related medical issues in specific geographic areas (e.g. outbreaks, epidemics, Lyme Disease, Legionnaires Disease, etc).

Another illustrated embodiment of the present invention provides the ability to update diagnostic, treatment and pharmaceutical databases and search algorithms system wide using encrypted data and controlled software approval and release methodologies.

Another illustrated embodiment of the present invention provides a methodology for storing patient data and utilizing both currently available and historical patient data in making a diagnosis or in identifying trends that may be detrimental to the health of the patient.

Another illustrated embodiment of the present invention provides a methodology for processing, analyzing, classifying, correlating, recognizing, characterizing and/or comparing heart beat, pulse data and/or breathing sounds or other data to identify signs, symptoms, latent or potential anomalies, abnormal characteristics and/or trends that may require further investigation or treatment.

Another illustrated embodiment of the present invention provides a method for continuously monitoring patient sensor data while the patient is being treated, transported or is under care in a facility, hospital, emergency room or Intensive Care Unit (ICU) and continuously evaluating the patient's condition based upon the collected and analyzed data. Should the patient's data exceed approved medical standards, the system should take predetermined actions including alerting on-duty medical personnel.

Another illustrated embodiment of the present invention provides a methodology for using a Certified Self Test Unit (CSTU) to ensure that the basic sensor suite is correctly calibrated and all sensors are reading within specified parameters.

Such embodiments are in contrast to conventional techniques for identifying, diagnosing and treating the illness, malady, disease, infection, condition or trauma afflicting the patient. In particular, using conventional techniques, identifying, diagnosing and treating illnesses, diseases, infections or trauma must be done by or under the direction or supervision of licensed and certified medical doctors or specialists, whereas these embodiments may utilize a trained operator such as an EMT, nurse, paramedic or corpsman without the participation, supervision or intervention of a medical doctor or specialist. .

A more detailed description of the systems, apparatus and methods for gathering, processing, analyzing, classifying, recognizing, characterizing and/or comparing patient data and utilizing the results to make a unique and unambiguous or a high confidence informed decision on a diagnosis and the associated treatment regimen is now described.

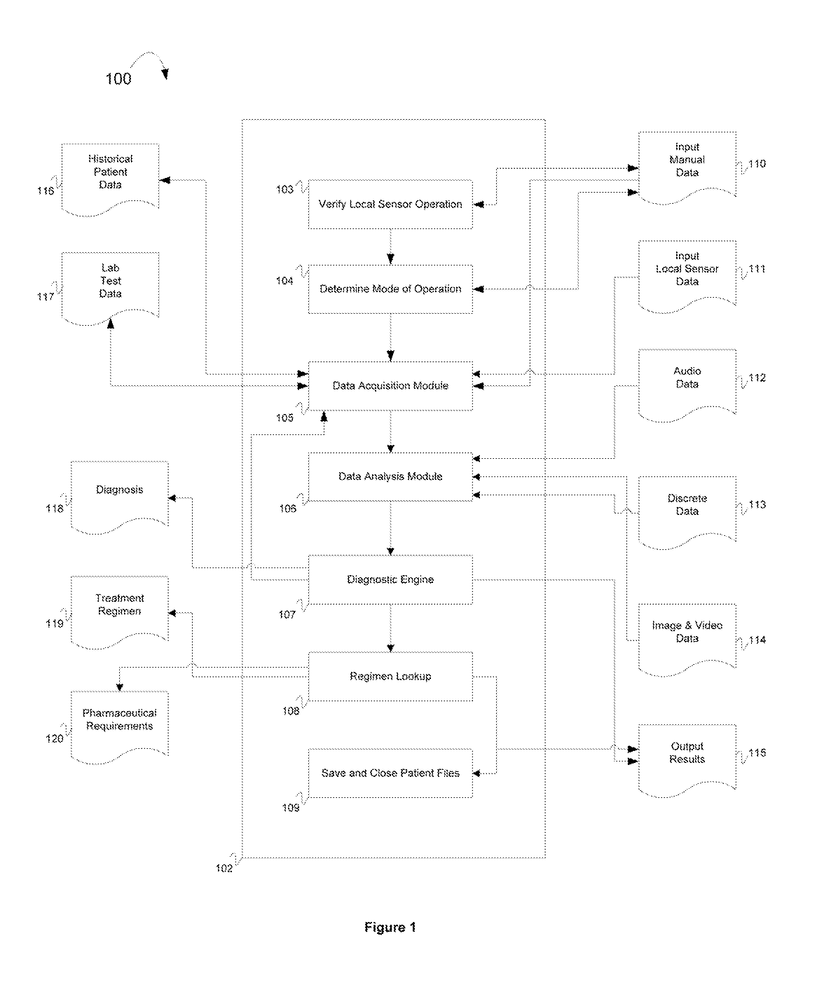

FIG. 1 is a system diagram for a medical analysis and diagnostic system, according to some example embodiments. FIG. 1 illustrates a system 100 that includes a medical analysis and diagnostic system. The medical analysis and diagnostic system 102 may be a mobile system or a fixed base client/server system serving both local and remote systems. In some example embodiments, the medical analysis and diagnostic system 102 may operate in a semi-autonomous manner without being directly connected to additional laboratory test equipment. In other example embodiments, the medical analysis and diagnostic system 102 may operate in a semi-autonomous manner and may or may not be directly connected to additional laboratory test equipment. Moreover, as further stated below, the various modules of the medical analysis and diagnostic system may all reside within a single processing unit.

Medical analysis and diagnostic system 102 comprises a sensor verification module 103, a mode of operation module 104, a data acquisition module 105, a data analysis module 106, a diagnostic engine 107, a regimen lookup module 108 and a data retention module 109. Mode of operation 104 receives manual inputs 110 to identify and verify the operator, determine the mode of operation and uniquely identify the patient. Data acquisition module 105 receives additional manual inputs 110 to provide unique identification of the patient, chief complaint(s) and other patient information, local sensor data 111, historical patient data 116 if available and lab test data 117 if requested and available. Data acquisition module 105 will then pass the collected data onto the data analysis module 106 for further processing. Data analysis module 106 will process, analyze, classify, correlate, characterize, recognize and/or compare audio data 112, discrete data 113, image and video data 114 and any other data types, files and media collected from the manual inputs 110, local sensor data 111, historical patient data 116 and lab test data 117 as it becomes available and utilize it to identify any signs, symptoms, potential anomalies or abnormal characteristics and produce search criteria suitable for use in the diagnostic engine 107. It is understood that the data analysis module 106 may consist of hardware, software and/or firmware components or a mixture thereof. The diagnostic engine or engines 107 may consist of one or more expert systems, state machines or other methodologies and utilizes all available search criteria derived from currently available geographic and point in time information, patient chief complaint(s), patient interviews, processed patient sensor data, processed patient inputted data and any available patient historical data to search a diagnostic database that is populated with all known illnesses, diseases, infections and traumas, along with their associated data, signs and symptoms, and generate a unique and unambiguous diagnosis or a high confidence informed decision on a diagnosis of the specific illness, malady, disease, infection, condition or trauma afflicting the patient. If the diagnostic engine 107 is able to identify a unique and unambiguous diagnosis, then this diagnosis 118 will be selected. Otherwise, if a high confidence informed decision on a diagnosis can be made, then this diagnosis 118 will be selected. If the diagnosis is ambiguous and not high confidence, then the diagnostic engine 107 will determine additional tests to remove the ambiguity and/or increase the confidence factor and pass this information back to the data acquisition module 105. Once an unambiguous or high confidence diagnosis 118 is identified, the diagnostic engine 107 will pass that information to the regimen lookup module 108, which will identify the corresponding treat regimen 119 and any associated pharmaceutical requirements 120. The regimen lookup module 108 will then pass the diagnosis 118, the corresponding treatment regimen 119 and any associated pharmaceutical requirements 120 to output results 115 to be made available to the operator and/or the patient. Save and close patient files 109 is then accomplished and the analysis and diagnostic session is ended.

Operations, according to example embodiments, are now described. In certain embodiments, the operations are performed by instructions residing on machine-readable media (e.g., software or firmware), while in other embodiments, the methods are performed by hardware or other logic (e.g., digital logic).

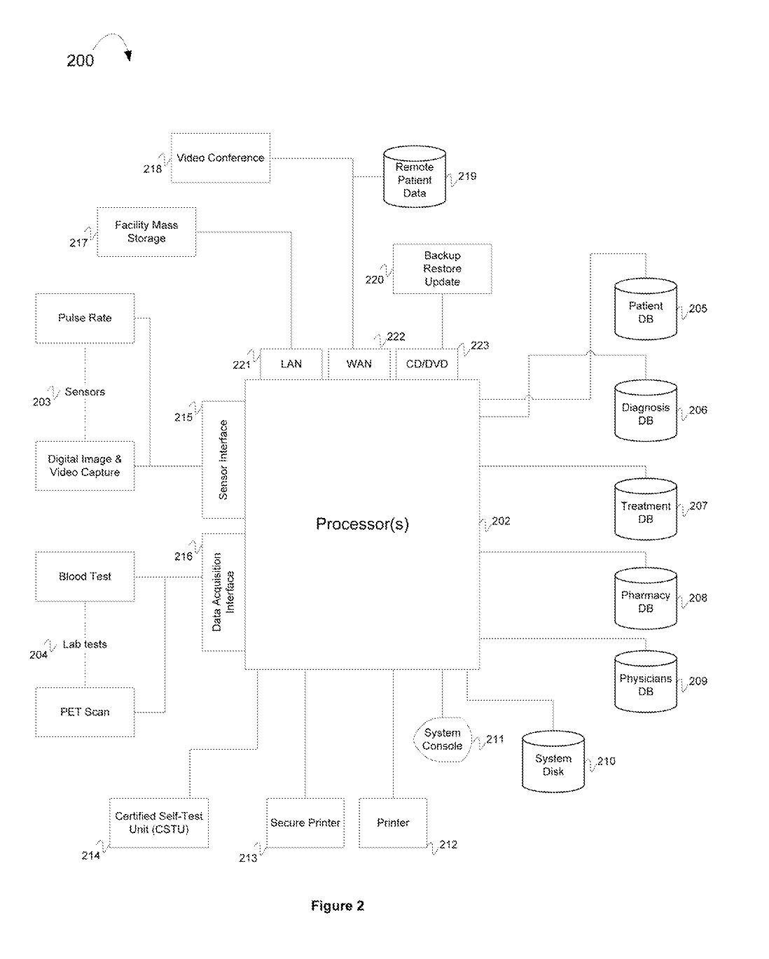

FIG. 2 is a detailed block diagram for a computerized semiautonomous medical analysis and diagnostic system, according to some example embodiments, and is now described. In particular, FIG. 2 illustrates a computerized semiautonomous medical analysis and diagnostic system that may be used in a standalone mobile or facility environment, according to some example embodiments. As illustrated in FIG. 2, the computer system 200 comprises processor(s) 202 which also includes any necessary memory, internal bus, input/output controllers, various interfaces, one or more disk drive(s), one or more database(s), storage facilities, sensors, network connections, printers, console(s) and a certified self test unit. The processor(s) 202 may comprise any suitable processor architecture. The computerized semiautonomous medical analysis and diagnostic system 200 may comprise one, two, three, or more processors, any of which may execute a set of instructions in accordance with embodiments of the invention.

Various local analog, digital or digitizing sensors 203 are utilized to collect analog, digital, discrete, pressure, audio, high definition color and/or grayscale image and video, and/or other data types and convert this data to a format suitable for uploading to the mobile computer system through interface 215 for further processing, analyzing, classifying, correlating, characterizing, pattern recognition and/or comparing, and generation of search criteria suitable for use with the diagnostic search engine, according to some example embodiments.

Laboratory test equipment 204 may or may not be connected through interface 216 to download analog, digital, discrete, pressure, audio, image and/or video data, and other data types, files and media as they become available for further processing, analyzing, classifying, correlating, characterizing and/or pattern recognition, comparing and generation of search criteria suitable for use with the diagnostic search engine. It will be understood by those skilled in the art that interfaces 215 and 216 may be implemented using LAN, WAN, USB, Bluetooth, wireless, cellular, proprietary or other network communication protocols, or a combination thereof in order to maximize connectivity, efficiency and throughput, according to some example embodiments.

According to some example embodiments, one or more databases may be implemented to provide access to required information. Patient database 205 will contain all available local data and files on the patient currently being examined or treated. The diagnostic database 206 will contain the most currently available medical information on all known illnesses, diseases, infections, traumas and other maladies. The treatment database 207 will contain the most currently available recommended treatment regimens associated with the illnesses, diseases, infections, traumas and other maladies contained in the diagnostic database 206, including whether over-the-counter or prescription pharmaceuticals are indicated as part of the treatment regimen. The pharmacy database 208 will contain the most currently available list of over-the-counter and prescription pharmaceuticals and if they are indicated as part of the treatment regimen, the patient's digital folder or record will be accessed to determine if there are any known redundancies, drug reactions, allergies or potential interactions with other prescribed medications. The physician database 209 will contain the most currently available list of medical doctors and specialists by specialty and geographic area and will be accessed in the event that referral to a medical doctor or specialist is required. It will be understood by those skilled in the art that two or more of these databases may be consolidated into a single database.

The system console 211 may be a console, keyboard, touch screen or other manual input device and is used for system dialog and maintenance functions, as well as a data acquisition module to input manual inputs to provide unique identification of the patient, chief complaint(s) and other patient information. System disk 210 holds all operating system and application software, according to some example embodiments. Printer 212 may be used to print off patient information, diagnosis, treatment regimens, pharmaceuticals and any other required information, according to some example embodiments. Secure printer 213 is utilized to print off prescriptions and other secure documents as required, according to some example embodiments.

It will be understood by those skilled in the art that interfaces 221, 222 and 223 may be implemented using LAN, WAN, USB, Bluetooth, wireless, cellular, proprietary or other network communication protocols, or a combination thereof in order to maximize connectivity, efficiency and throughput, and may be connected to remote patient data files 219, backup, restore or update 220, facility mass storage 217, or allow for video conferencing 218, according to some example embodiments.

A certified self test unit 214 may be implemented in order to ensure that the local sensor suite is correctly calibrated and all sensors are reading within specified parameters, according to some example embodiments.

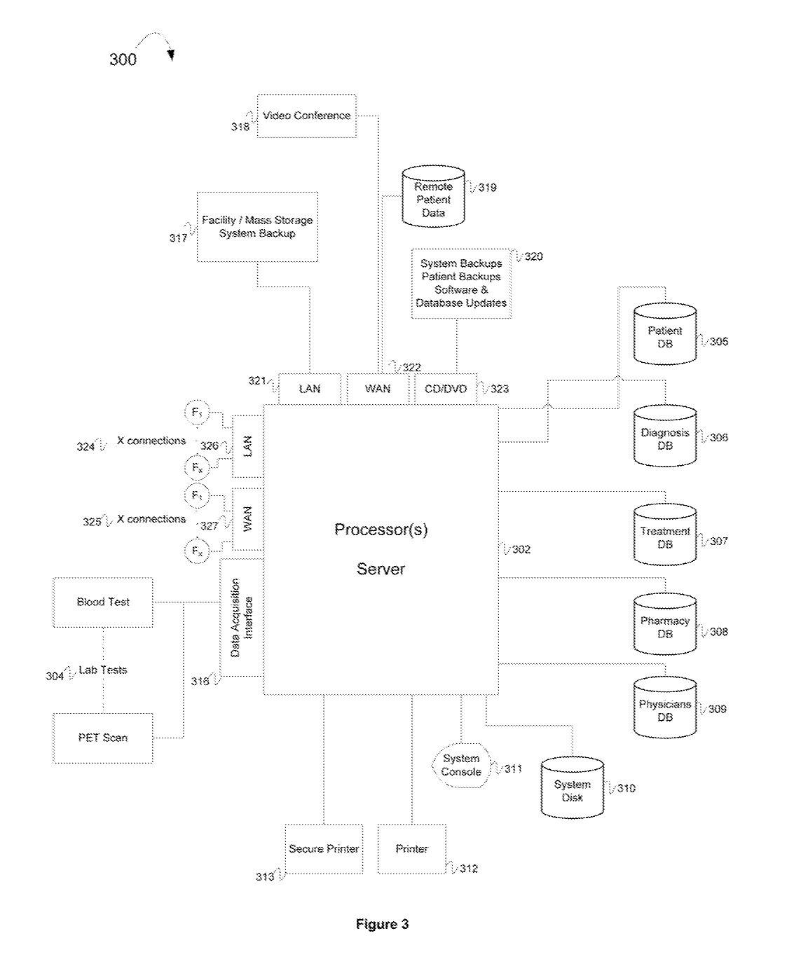

FIG. 3 is a detailed block diagram for a computerized semiautonomous medical analysis and diagnostic system, according to some example embodiments, and is now described. In particular, FIG. 3 illustrates a computerized semiautonomous medical analysis and diagnostic system that may be used as the server in a facility or remote distributed (client/server) environment, according to some example embodiments. As illustrated in FIG. 3, the computer system 300 comprises processor(s) 302 which also includes any necessary memory, internal bus, input/output controllers, various interfaces, one or more disk drive(s), one or more database(s), storage facilities, sensors, network connections, printers, console(s) and a self test unit. The processor(s) 302 may comprise any suitable processor architecture. The computerized semiautonomous medical analysis and diagnostic system 300 may comprise one, two, three, or more processors, any of which may execute a set of instructions in accordance with embodiments of the invention.

Multiple local or remote client systems 324 and 325 may be connected to the server through interfaces 326 and 327 for downloading client sensor analog, digital, discrete, pressure, audio, high definition color and/or grayscale image and/or video, and/or other data types for further processing, analyzing, classifying, characterizing, pattern recognition and/or comparing, and generating search criteria suitable for use with the diagnostic search engine, according to example embodiments.

Laboratory test equipment 304 may or may not be connected through interface 316 to download analog, digital, discrete, audio, pressure, image and/or video data, and/or other data types, files and media as they become available for further processing, analyzing, classifying, characterizing, pattern recognition and/or comparing, and generating of search criteria suitable for use with the diagnostic search engine. It will be understood by those skilled in the art that interfaces 316, 326 and 327 may be implemented using LAN, WAN, USB, Bluetooth, wireless, cellular, proprietary or other network communication protocols, or a combination thereof in order to maximize connectivity, efficiency and throughput, according to some example embodiments.

According to some example embodiments, one or more databases may be implemented to provide access to required information. Patient database 305 will contain all available data and files on the patient currently being examined or treated. The diagnostic database 306 will contain the most currently available medical information on all known illnesses, diseases, infections, traumas and other maladies. The treatment database 307 will contain the most currently available recommended treatment regimens associated with the illnesses, diseases, infections, traumas and other maladies contained in the diagnostic database 306, including whether over-the-counter or prescription pharmaceuticals are indicated as part of the treatment regimen. The pharmacy database 308 will contain the most currently available list of over-the-counter and prescription pharmaceuticals and if they are indicated as part of the treatment regimen, the patient's digital folder or record will be accessed to determine if there are any known redundancies, drug reactions, allergies or potential interactions with other prescribed medications. The physician database 309 will contain the most currently available list of medical doctors and specialists by specialty and geographic area and will be accessed in the event that referral to a medical doctor or specialist is required. It will be understood by those skilled in the art that two or more of these databases may be consolidated into a single database.

After the diagnostic session is complete, any results, including required patient information, diagnosis, treatment regimens, pharmaceuticals and any other information is passed back to the appropriate local or remote client system 324 or 325 through interface 326 or 327, according to some example embodiments.

The system console 311 may be a console, keyboard, touch screen or other manual input device and is used for system dialog and maintenance functions, as well as a data acquisition module to input manual inputs to provide unique identification of the patient, chief complaint(s) and other patient information. System disk 310 holds all operating system and application software, according to some example embodiments. Printer 312 may be used to print off patient information, diagnosis, treatment regimens and any other required information, according to some example embodiments. Secure printer 313 is utilized to print off prescriptions and other secure documents as required, according to some example embodiments.

It will be understood by those skilled in the art that interfaces 321, 322 and 323 may be implemented using LAN, WAN, USB, Bluetooth, wireless, cellular, proprietary or other network communication protocols, or a combination thereof in order to maximize connectivity, efficiency and throughput, and may be connected to remote patient data files 319, backup, restore or update 320 facility mass storage 317, or allow for video conferencing 318, according to some example embodiments.

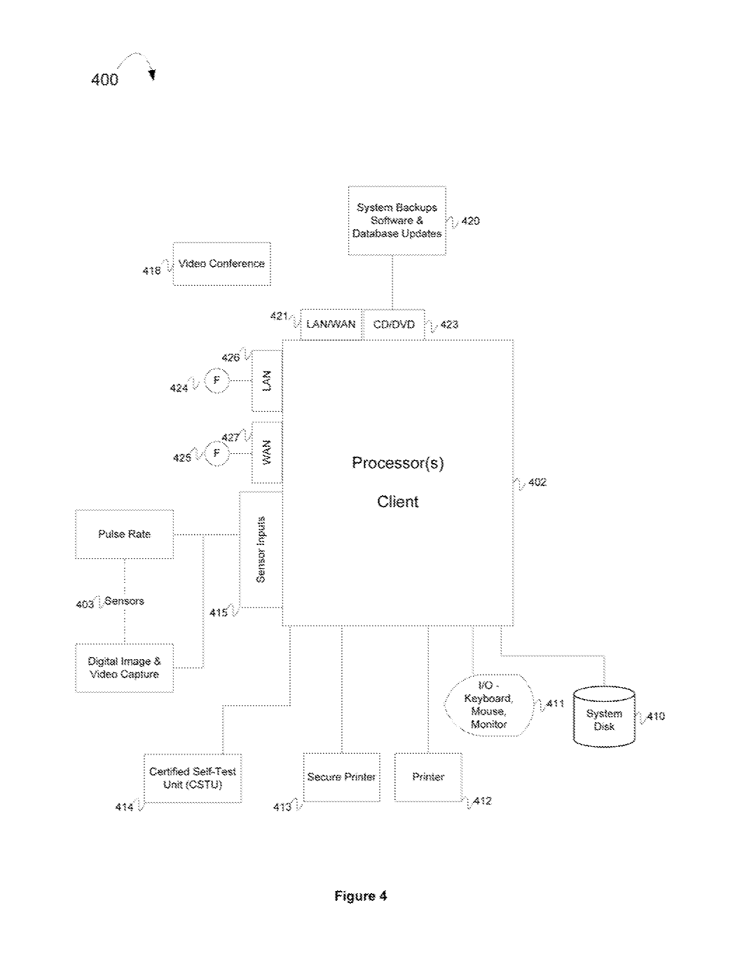

FIG. 4 is a detailed block diagram for a computerized semiautonomous medical analysis and diagnostic system, according to some example embodiments, and is now described. In particular, FIG. 4 illustrates a computerized semiautonomous medical analysis and diagnostic system that may be used as the client in a facility or remote distributed (client/server) environment, according to some example embodiments. As illustrated in FIG. 4, the computer system 400 comprises processor(s) 402 which also includes any necessary memory, internal bus, input/output controllers, various interfaces, one or more disk drive(s), one or more database(s), storage facilities, sensors, network connections, printers, console(s) and a self test unit. The processor(s) 402 may comprise any suitable processor architecture. The computerized semiautonomous medical analysis and diagnostic system 400 may comprise one, two, three, or more processors, any of which may execute a set of instructions in accordance with embodiments of the invention.

According to some sample embodiments, various analog, digital or digitizing sensors 403 may be utilized to collect analog, digital, discrete, audio, pressure, image, video and/or other data types and converting this data to a format suitable for uploading to the client computer system through interface 415 for further processing, analyzing, classifying, characterizing, pattern recognition and/or comparing, and generation of search criteria suitable for use with the diagnostic search engine, according to some example embodiments.

The client system may be connected to server 424 or 425 through interface 426 or 427 for uploading client sensor analog, digital, discrete, audio, pressure, high definition color and/or grayscale image and video and/or other data types to the server for further processing, analyzing, classifying, characterizing, pattern recognition and/or comparing, and generation of search criteria suitable for use with the diagnostic search engine, according to example embodiments. It will be understood by those skilled in the art that interfaces 415, 426 and 427 may be implemented using LAN, WAN, USB, Bluetooth, wireless, cellular, proprietary or other network communication protocols, or a combination thereof in order to maximize connectivity, efficiency and throughput, according to some example embodiments.

After the diagnostic process is complete, any required patient information, diagnosis, treatment regimens, pharmaceuticals and any other required information is downloaded from the server back to the appropriate local or remote client system 424 or 425 through interface 426 or 427, according to some example embodiments.

The system console 411 may be a console, keyboard, touch screen or other manual input device and is used for system dialog and maintenance functions, as well as a data acquisition module to input manual inputs to provide unique identification of the patient, chief complaint(s) and other patient information. System disk 410 holds all operating system and application software, according to some example embodiments. Printer 412 may be used to print off patient information, diagnosis, treatment regimens and any other required information, according to some example embodiments. Secure printer 413 is utilized to print off prescriptions and other secure documents as required, according to some example embodiments.

It will be understood by those skilled in the art that interfaces 421 and 423 may be implemented using LAN, WAN, USB, Bluetooth, wireless, cellular, proprietary or other network communication protocols, or a combination thereof in order to maximize connectivity, efficiency and throughput, and may be connected to backup, restore or update 420 or allow for video conferencing 418, according to some example embodiments.

A certified self test unit 414 may be implemented in order to ensure that the basic sensor suite is correctly calibrated and all sensors are reading within specified parameters, according to some example embodiments.

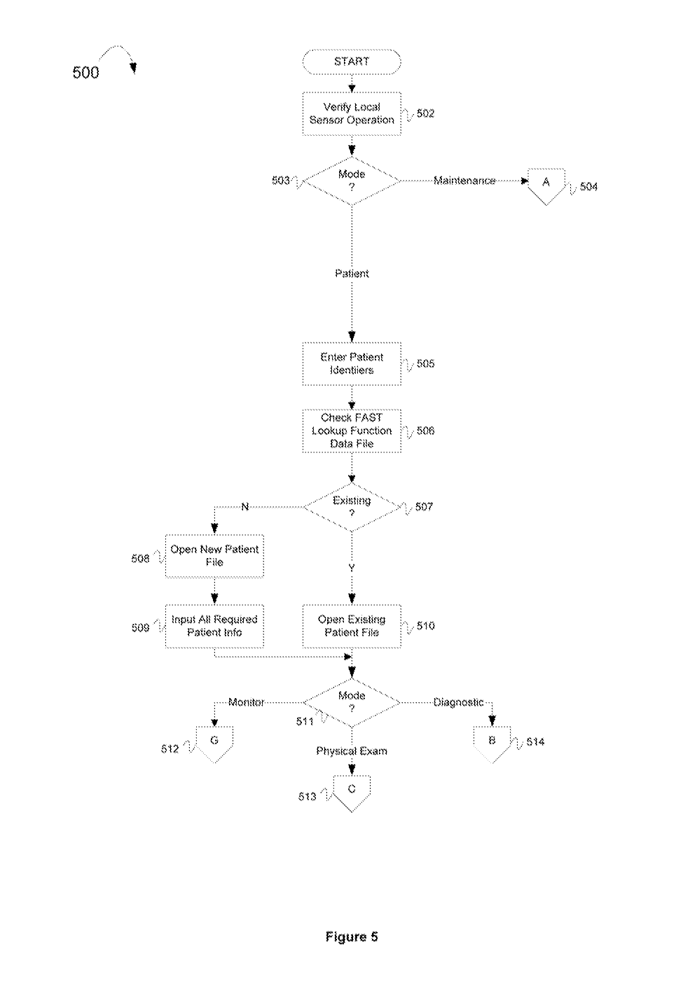

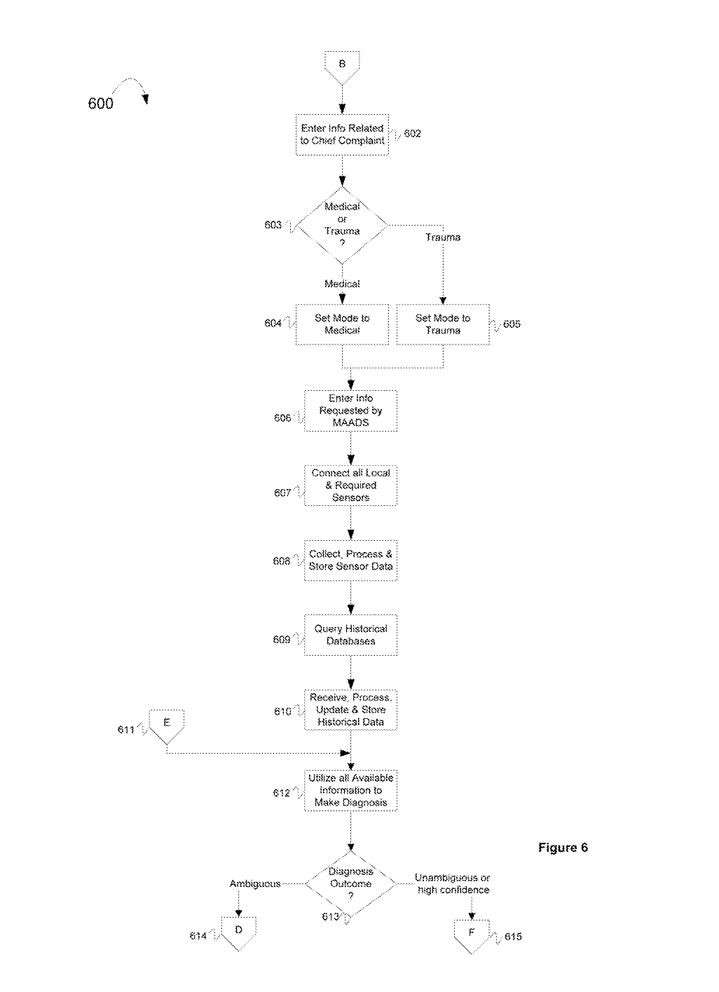

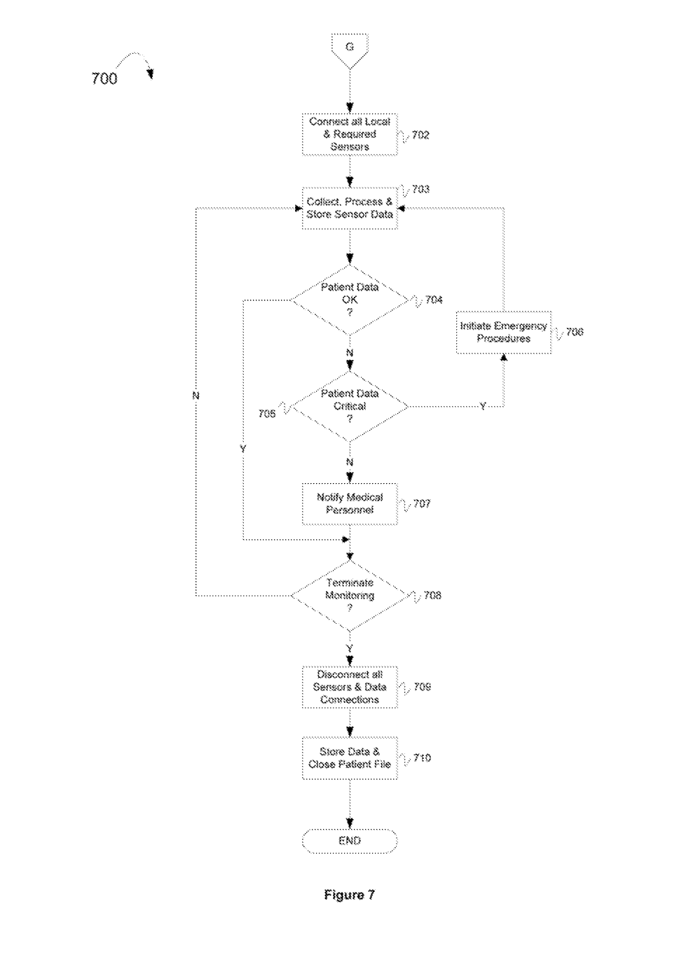

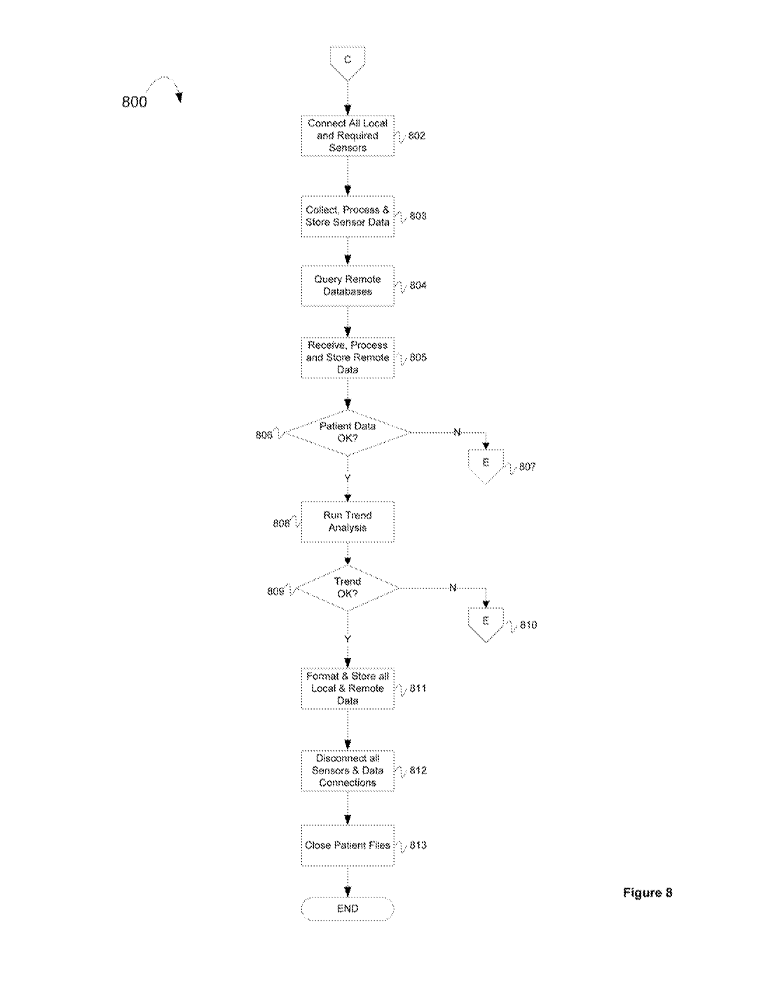

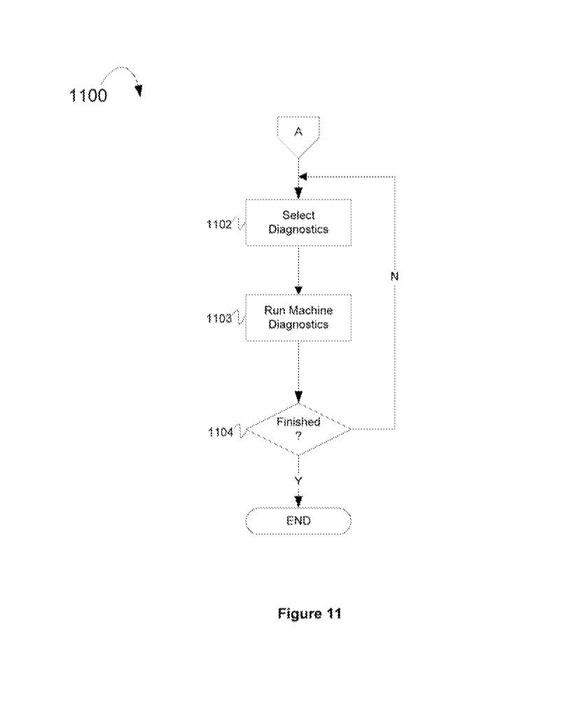

A method 500 is described with reference to FIG. 5. In some sample embodiments, FIG. 5 is a diagram of a method for a medical analysis and diagnostic system that includes block 502 for verifying local sensor operation; block 503 for determining the mode of operation as either maintenance or patient; if mode of operation is maintenance at block 503 then proceed to FIG. 11(A) 504; if mode of operation is patient then entering patient identifiers at block 505 to determine if this is a new or existing patient 507; either opening a new patient file at block 508 and populating it at block 509 or opening the existing patient file at block 510; determining the mode of operation as either monitoring at block 511 then proceed to FIG. 7(G) 512, performing a physical examination at block 511 then proceed to FIG. 8(C) 513 or performing diagnostics on the patient 511 then proceed to FIG. 6(B) 514, according to some example embodiments.

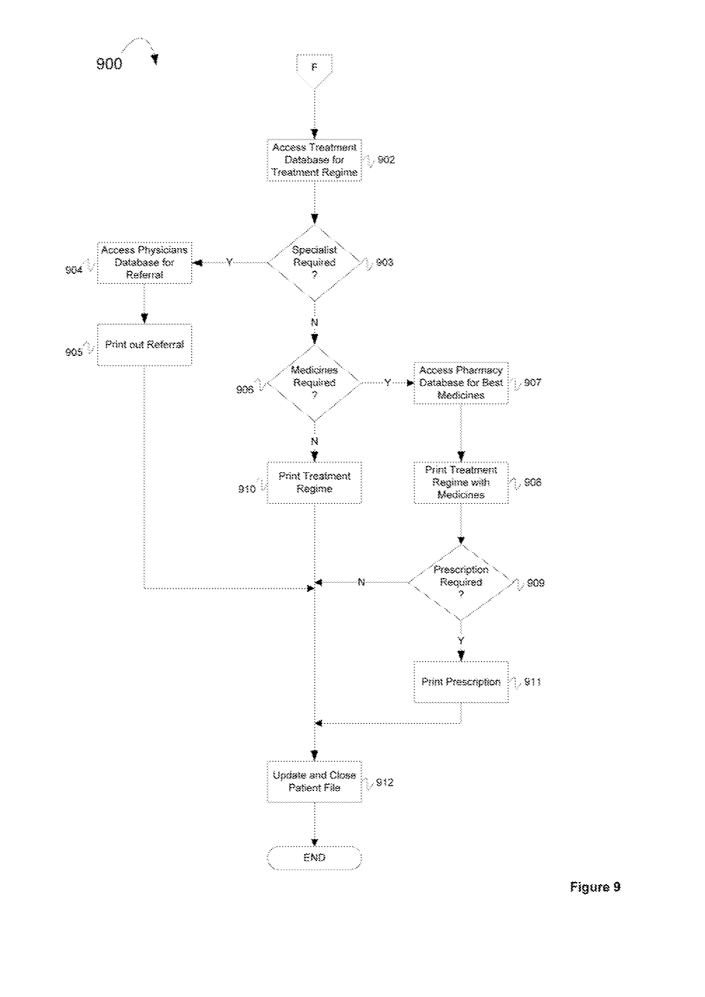

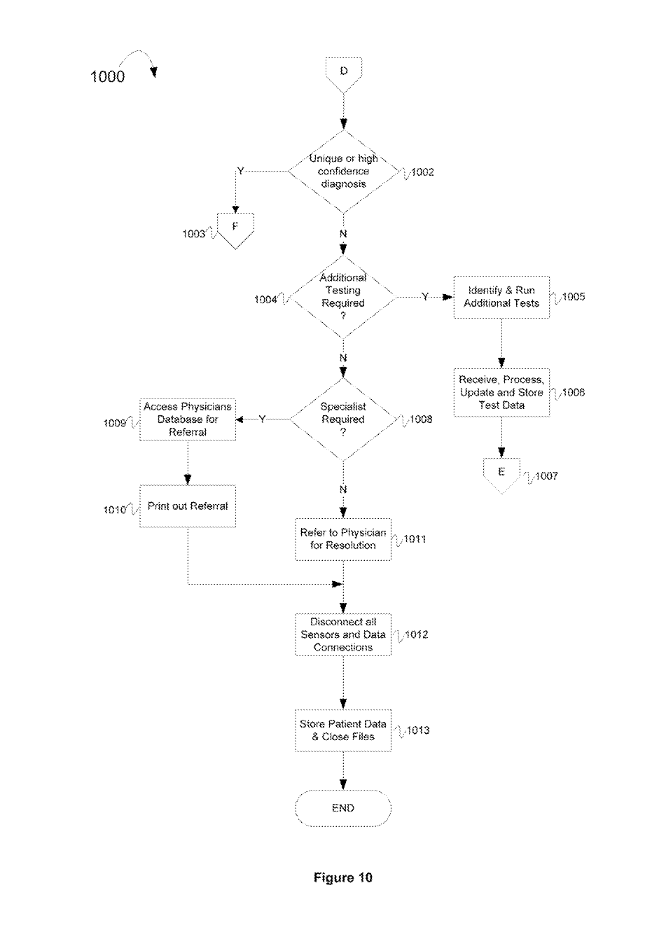

A method 600 is described with reference to FIG. 6. In some sample embodiments, FIG. 6 is a diagram of a method for a medical analysis and diagnostic system diagnostic mode that includes acquiring patient information including unique identification of the patient, chief complaint(s) 602; determining whether the problem is medical or trauma related 603 and setting the mode to medical 605 or trauma 605; performing the patient interview, updating or storing the patient information 606; connecting all currently available local and required sensors to the patient 607; collecting, storing, processing, analyzing, classifying, comparing, recognizing and correlating the currently available local sensor and laboratory test data to generate search criteria suitable for use in the diagnostic search engine 608; importing, processing, analyzing, classifying, comparing, recognizing and correlating patient test results from other sources to generate search criteria suitable for use in the diagnostic search engine 612; locating, retrieving, processing, analyzing, classifying, comparing, recognizing and correlating historical data related to the patient to generate search criteria suitable for use in the diagnostic search engine 609; utilizing all available patient information, currently available local sensor and laboratory data search criteria, imported test results search criteria and historical patient data search criteria to query a diagnostic database and make a diagnosis 612; determining whether the diagnosis is ambiguous or unambiguous 613; proceeding to FIG. 10(D) 614 if the diagnosis is ambiguous; or proceeding to FIG. 9(F) 615 if the diagnosis is unambiguous or a high confidence diagnosis, according to some example embodiments.

A method 700 is described with reference to FIG. 7. In some sample embodiments, FIG. 7 is a diagram of a method for a medical analysis and diagnostic system monitoring mode that includes connecting all local and required sensors 702; collecting, processing, analyzing, classifying, comparing, recognizing, correlating and/or comparing the currently available local sensor and laboratory test data 703 to determine if patient data is within established parameters 704 and, if so, check to see if monitoring is still required 708; if patient data is outside parameters and critical, initiate emergency procedures 706; if patient data is outside parameters and not critical, notify medical personnel 707; if monitoring is no longer required 708, disconnect all sensors and data connections 709; store patient data and close patient files 710, according to some example embodiments.

A method 800 is described with reference to FIG. 8. In some sample embodiments, FIG. 8 is a diagram of a method for a medical analysis and diagnostic system physical examination mode that includes connecting all local and required sensors 802; collecting, processing, analyzing, classifying, recognizing, comparing and/or correlating the currently available local sensor and laboratory test data 803; querying any remote databases 804; receiving, processing, analyzing, classifying, recognizing, correlating and/or comparing the local and remote data 805; determining if patient data is within established parameters 806 and if not within established parameters begin diagnostic mode 807 at FIG. 6(E); if patient data is okay then run a trend analysis 808; if trend analysis is not okay 809 then begin diagnostic mode 810 at FIG. 6(E); if trend analysis is okay then format and store all patient data 811; disconnect all sensors and data connections 812; and close patient files 813, according to some example embodiments.

A method 900 is described with reference to FIG. 9. In some sample embodiments, FIG. 9 is a diagram of a method for a medical analysis and diagnostic system treatment determination mode that includes accessing a treatment database 902; determining if a medical specialist is required 903 and if so, identifying a medical specialist 904 and making a referral 905; if a medical specialist is not required then determining if medications are required 906; if medications are not required then printing out the treatment regime 910; if medications are required then accessing a pharmaceutical database 907 to determine which medications are the most beneficial drug or drugs available to treat the diagnosed illness, malady, disease, infection, condition or trauma; printing out the treatment regime with medications 908; if a prescription is required 909 then print out the prescription 911; then storing patient data and closing patient files 912, according to some example embodiments.

A method 1000 is described with reference to FIG. 10. In some sample embodiments, FIG. 10 is a diagram of a method for a medical analysis and diagnostic system which is a continuation of the diagnostic mode that includes determining whether the diagnostic result is unique or a high confidence diagnosis 1002 and if so it proceeds to FIG. 9(F) 1003 to determine the appropriate treatment regimen; if the diagnostic result is not a unique or high confidence diagnosis, then a determination is made as to whether additional testing would produce an unambiguous or high confidence result 1004 and if so, additional tests are identified and run 1005, test results are received, processed, updated and stored 1006, and proceeds to FIG. 6(E) 1007; if additional testing is not indicated then a determination is made as to whether medical specialist is required 1008 and if so, identifying a medical specialist 1009 and making a referral 1010; if a medical specialist is not required then referring to a medical doctor for a resolution 1011; disconnecting all sensors and data connections 1012; storing patient data and closing patient files 1013, according to some example embodiments.

A method 1100 is described with reference to FIG. 11. In some sample embodiments, FIG. 11 is a diagram of a method for a Medical Analysis and Diagnostic System maintenance mode that includes selecting the machine diagnostics to be run 1102, running the selected machine diagnostics 1103, and determining if more diagnostics need to be run 1104, according to some example embodiments.

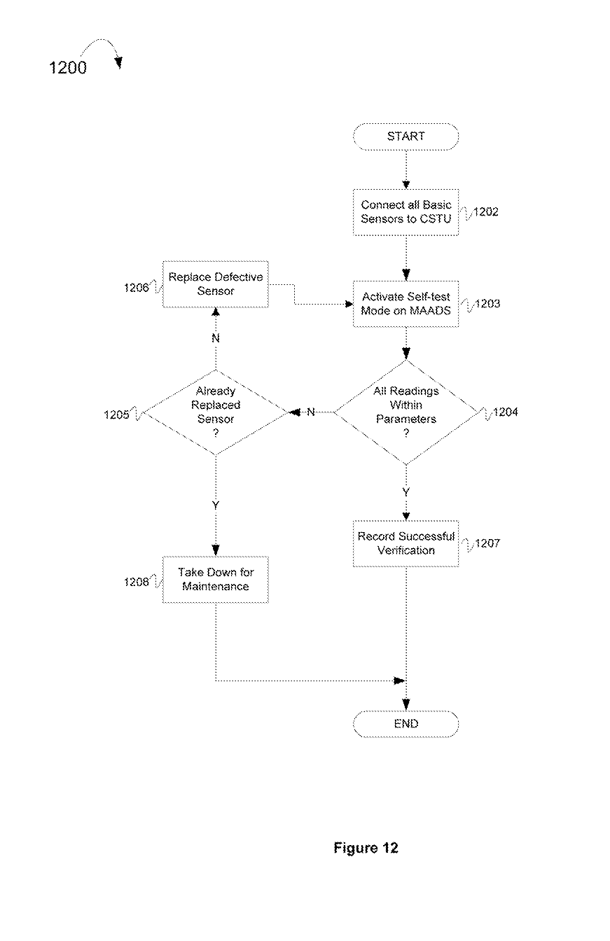

A method 1200 is described with reference to FIG. 12. In some sample embodiments, FIG. 12 is a diagram of a method for a medical analysis and diagnostic system mode for verification of sensor operation that includes connecting all basic sensors to a certified self test unit 1202; activating the self test mode 1203; determining whether all readings are within preset parameters 1204 and if so, record a successful verification 1207; if all readings are not within preset parameters then determining if the sensor in question has already been replaced 1205 and if so, taking the system down for maintenance 1208; if all readings are not within preset parameters and the sensor in question has not already been replaced, then replacing the defective sensor 1206 and repeating the test, according to some example embodiments.

In the foregoing description, numerous specific details such as logic implementations, opcodes, means to specify operands, resource partitioning, sharing, and/or duplication implementations, types and interrelationships of system components, and logic partitioning/integration choices are set forth in order to provide a more thorough understanding of the present invention. It will be appreciated, however, by one skilled in the art that embodiments of the invention may be practiced without such specific details. In other instances, control structures, gate level circuits and full software instruction sequences have not been shown in detail in order not to obscure the embodiments of the invention. Those of ordinary skill in the art, with the included descriptions will be able to implement appropriate functionality without undue experimentation.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Embodiments of the invention include features, methods or processes that may be embodied within machine-executable instructions provided by a machine-readable medium. A machine-readable medium includes any mechanism which provides (i.e., stores and/or transmits) information in a form accessible by a machine (e.g., a computer, a network device, a personal digital assistant, manufacturing tool, any device with a set of one or more processors, etc.). In example embodiments, a machine-readable medium includes volatile and/or non-volatile media (e.g., read only memory (ROM), random access memory (RAM), magnetic disk storage media, optical storage media, flash memory devices, etc.).

Such instructions are utilized to cause a general purpose or special purpose processor, programmed with the instructions, to perform methods or processes of the embodiments of the invention. Alternatively, the features or operations of embodiments of the invention are performed by specific hardware components which contain hard-wired logic for performing the operations, or by any combination of programmed data processing components and specific hardware components. Embodiments of the invention include software, data processing hardware, data processing system-implemented methods, and various processing operations, further described herein.

In view of the wide variety of permutations to the embodiments described herein, this detailed description is intended to be illustrative only, and should not be taken as limiting the scope of the invention. What are claimed as the invention, therefore, are all such modifications as may come within the scope and spirit of the following claims and equivalents thereto. Therefore, the specification and drawings are to be regarded in an illustrative rather than a restrictive sense.

What is claimed is:

1. A computerized method comprising:
diagnosing a patient, wherein the diagnosing comprises:
receiving a patient identification of the patient;
determining, using one or more sensors, one or more current body characteristics of the patient comprising at least one of pulse rate, body temperature, blood pressure, respiration, and skin condition;
creating a current multimedia representation for each of the one or more current body characteristics determined by using the sensor;
comparing the current multimedia representation to previous multimedia representations of each of the one or more body characteristics from other persons;
selecting a diagnosis and a diagnosis confidence factor for the diagnosis for the patient based on the comparing of the current multimedia representation to the previous multimedia representations of each of one or more the body characteristics;
determining whether the diagnosis confidence factor exceeds a high confidence factor threshold;
in response to the diagnosis confidence factor not exceeding the high confidence factor threshold, selecting a different current body characteristic of the patient to determine to increase the diagnosis confidence factor; and
in response to the diagnosis confidence factor exceeding the high confidence factor threshold, selecting the diagnosis for the patient.

2. The computerized method of claim 1,
wherein diagnosing the patient comprises downloading remote patient data from a remote server based on the patient identification, and
wherein selecting the diagnosis and the diagnosis confidence factor for the diagnosis for the patient is based on the remote patient data.

3. The computerized method of claim 1,
wherein diagnosing the patient comprises retrieving patient historical data that comprises past body characteristics of the patient that were determined at a prior time, wherein the past body characteristics of the patient comprises at least one of pulse rate, body temperature, blood pressure, respiration, and skin condition, and
wherein selecting the diagnosis and the diagnosis confidence factor for the diagnosis for the patient is based on the patient historical data.

4. The computerized method of claim 1, wherein diagnosing the patient comprises:
in response to the diagnosis confidence factor exceeding the high confidence factor threshold, selecting a treatment that is derived from the diagnosis for the patient.

5. The computerized method of claim 4, wherein diagnosing the patient comprises:
in response to selecting the treatment, determining whether a treatment medication is to be administered as part of the treatment;
in response to the medication to be administered as part of the treatment, accessing a list of current medications being used by the patient and allergies of the patient;
determining whether the current medications and the allergies of the patient conflict with the treatment medication; and
in response to the current medications and the allergies of the patient are not in conflict with the treatment medication, authorizing use of the treatment medication for the patient.

6. The computerized method of claim 1, wherein the other persons comprises persons in a same geographic area as the patient.

7. The computerized method of claim 1, wherein creating the current multimedia representation comprises creating at least one of a video representation, an audio representation, an image representation, and a waveform representation.

8. A computer program product for presenting media content, the computer program product comprising:
a computer readable storage medium having computer usable program code embodied therewith, the computer usable program code comprising a computer usable program code configured to:
diagnose a patient, wherein the computer usable program code configured to diagnose comprises computer usable program code configured to:
receive a patient identification of the patient;
determine, using one or more sensors, one or more current body characteristics of the patient comprising at least one of pulse rate, body temperature, blood pressure, respiration, and skin condition;
create a current multimedia representation for each of the one or more current body characteristics determined by using the sensor;
compare the current multimedia representation to previous multimedia representations of each of the one or more body characteristics from other persons;
select a diagnosis and a diagnosis confidence factor for the diagnosis for the patient based on the comparing of the current multimedia representation to the previous multimedia representations of each of one or more the body characteristics;
determine whether the diagnosis confidence factor exceeds a high confidence factor threshold;
in response to the diagnosis confidence factor not exceeding the high confidence factor threshold, select a different current body characteristic of the patient to determine to increase the diagnosis confidence factor; and
in response to the diagnosis confidence factor exceeding the high confidence factor threshold, select the diagnosis for the patient.

9. The computer program product of claim 8,
wherein the computer usable program code configured to diagnose the patient comprises computer usable program code configured to download remote patient data from a remote server based on the patient identification, and
wherein the computer usable program code configured to select the diagnosis and the diagnosis confidence factor for the diagnosis for the patient is based on the remote patient data.

10. The computer program product of claim 8,
wherein the computer usable program code configured to diagnose the patient comprises computer usable program code configured to retrieve patient historical data that comprises past body characteristics of the patient that were determined at a prior time, wherein the past body characteristics of the patient comprises at least one of pulse rate, body temperature, blood pressure, respiration, and skin condition, and
wherein the computer usable program code configured to select the diagnosis and the diagnosis confidence factor for the diagnosis for the patient is based on the patient historical data.

11. The computer program product of claim 8, wherein the computer usable program code configured to diagnose the patient comprises computer usable program code configured to:
in response to the diagnosis confidence factor exceeding the high confidence factor threshold, select a treatment that is derived from the diagnosis for the patient.

12. The computer program product of claim 11, wherein the computer usable program code configured to diagnose the patient comprises computer usable program code configured to:
in response to selecting the treatment, determine whether a treatment medication is to be administered as part of the treatment;
in response to the medication to be administered as part of the treatment, access a list of current medications being used by the patient and allergies of the patient;
determine whether the current medications and the allergies of the patient conflict with the treatment medication; and
in response to the current medications and the allergies of the patient are not in conflict with the treatment medication, authorize use of the treatment medication for the patient.

13. The computer program product of claim 8, wherein the other persons comprises persons in a same geographic area as the patient.

14. The computer program product of claim 8, wherein the computer usable program code configured to create the current multimedia representation comprises computer usable program code to create at least one of a video representation, an audio representation, an image representation, and a waveform representation.

15. An apparatus comprising:
a processor; and
a computer readable storage medium having computer usable program code embodied therewith, the computer usable program code executable on the processor and configured to:
diagnose a patient, wherein the computer usable program code configured to diagnose comprises computer usable program code configured to:
receive a patient identification of the patient;
determine, using one or more sensors, one or more current body characteristics of the patient comprising at least one of pulse rate, body temperature, blood pressure, respiration, and skin condition;
create a current multimedia representation for each of the one or more current body characteristics determined by using the sensor;
compare the current multimedia representation to previous multimedia representations of each of the one or more body characteristics from other persons;
select a diagnosis and a diagnosis confidence factor for the diagnosis for the patient based on the comparing of the current multimedia representation to the previous multimedia representations of each of one or more the body characteristics;
determine whether the diagnosis confidence factor exceeds a high confidence factor threshold;
in response to the diagnosis confidence factor not exceeding the high confidence factor threshold, select a different current body characteristic of the patient to determine to increase the diagnosis confidence factor; and
in response to the diagnosis confidence factor exceeding the high confidence factor threshold, select the diagnosis for the patient.

16. The apparatus of claim 15,
wherein the computer usable program code configured to diagnose the patient comprises computer usable program code configured to download remote patient data from a remote server based on the patient identification, and
wherein the computer usable program code configured to select the diagnosis and the diagnosis confidence factor for the diagnosis for the patient is based on the remote patient data.

17. The apparatus of claim 15,
wherein the computer usable program code configured to diagnose the patient comprises computer usable program code configured to retrieve patient historical data that comprises past body characteristics of the patient that were determined at a prior time, wherein the past body characteristics of the patient comprises at least one of pulse rate, body temperature, blood pressure, respiration, and skin condition, and
wherein the computer usable program code configured to select the diagnosis and the diagnosis confidence factor for the diagnosis for the patient is based on the patient historical data.

18. The apparatus of claim 15, wherein the computer usable program code configured to diagnose the patient comprises computer usable program code configured to:
in response to the diagnosis confidence factor exceeding the high confidence factor threshold, select a treatment that is derived from the diagnosis for the patient.

19. The apparatus of claim 18, wherein the computer usable program code configured to diagnose the patient comprises computer usable program code configured to:
in response to selecting the treatment, determine whether a treatment medication is to be administered as part of the treatment;
in response to the medication to be administered as part of the treatment, access a list of current medications being used by the patient and allergies of the patient;
determine whether the current medications and the allergies of the patient conflict with the treatment medication; and
in response to the current medications and the allergies of the patient are not in conflict with the treatment medication, authorize use of the treatment medication for the patient.

20. The apparatus of claim 15, wherein the other persons comprises persons in a same geographic area as the patient.
